(19) United States
(12) Patent Application Publication
(10) Pub. No.: US 2025/0118786 A1
(43) Pub. Date: Apr. 10, 2025

(54) LONG-LIVED REDOX-ACTIVE MOLECULES WITH LOW REDOX POTENTIAL

(71) Applicant: President and Fellows of Harvard College, Cambridge, MA (US)

(72) Inventors: Min WU, Cambridge, MA (US); Roy G. GORDON, Cambridge, MA (US); Michael J. AZIZ, Cambridge, MA (US)

(21) Appl. No.: 18/684,449

(22) PCT Filed: Aug. 19, 2022
(86) PCT No.: PCT/US2022/040851

§ 371 Date: Feb. 16, 2024

Related U.S. Application Data
(60) Provisional application No. 63/234,957, filed on Aug. 19, 2021.

(51) Int. Cl. H01M 8/18; C07C 229/18; C07C 309/14

(52) U.S. Cl. CPC H01M 8/188; C07C 229/18; C07C 309/14

(57) ABSTRACT

The invention provides flow batteries and methods of using flow batteries including long-lived redox-active molecules with low redox potential.

### STATEMENT REGARDING FEDERALLY SPONSORED RESEARCH

This invention was made with government support under DE-AC05-76RLD1830, awarded by the Department of Energy. The government has certain rights in the invention.

### BACKGROUND OF THE INVENTION

The cost of electricity generated from renewable sources such as the sun and wind has become competitive with electricity derived from fossil fuels. Nonetheless, the widespread adoption of intermittent renewable electricity requires new methods for the reliable storage and delivery of electricity over long periods when these sources are unavailable for generation. Redox flow batteries (RFBs), whose energy and power capabilities can be scaled independently, may enable cost-effective long-duration discharge.

The all-vanadium redox flow battery chemistry is currently the most technologically developed but may not access much of the grid storage market due to electrolyte cost constraints. Emerging organic electrolytes comprising cheaper earth-abundant elements may address this limitation. However, organic electrolytes are more prone to molecular decomposition, which can lead to a progressive loss of charge storage capacity. Accordingly, there is a need for organic electrolytes with long term stability.

### SUMMARY OF THE INVENTION

The invention features redox flow batteries including long-lived redox active molecules.

In an aspect, the invention provides a compound of formula (I):

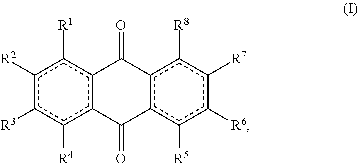

or an ion, salt, or hydroquinone thereof, where at least one of R¹, R², R³, R⁴, R⁵, R⁶, R⁷ and R⁸ is —N(R_{b})₂ or —N(R_{c})₂ wherein each R_{b} is independently an optionally substituted C₁₋₆ alkyl and each R_{c} is independently H or an optionally substituted C₁₋₆ alkyl, provided that when one R_{c} is H, the other R_{c} bound to the same N is an optionally substituted C₁₋₆ alkyl that is not —CH₂CH₂C(═O)OH; wherein at least one R_{b} or R_{c} is an optionally substituted C₁₋₆ alkyl including a —S(═O)₂Rₐ; —S(═O)₂ORₐ; —OS(═O)₂ORₐ; —P(═O)Rₐ₂; or P(═O)(ORₐ)₂ substituent or is an optionally substituted C₂₋₆ alkyl including a —C(═O)ORₐ substituent; and each remaining of R¹, R², R³, R⁴, R⁵, R⁶, R⁷ and R⁸ is independently selected from H; halo; optionally substituted C₁₋₆ alkyl; oxo; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —SRₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; and —P(═O)(ORₐ)₂; or any two adjacent groups selected from R⁸, R⁷, R⁶, and R⁵ are joined to form an optionally substituted 3-6 membered ring, where each Rₐ is independently H; optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group. The dashed bonds indicate full conjugation.

In some embodiments, any two or three of R¹, R², R³, R⁴, R⁵, R⁶, R⁷ and R⁸ are —N(R_{b})₂, e.g., R² and R⁶ or R² and R⁷. In some embodiments, each R_{b} is independently selected from a C₁₋₆ alkyl group substituted with S(═O)₂OH, OS(═O)₂OH, or C(═O)OH, such as —CH₂S(═O)₂OH, —CH₂CH₂S(═O)₂OH, —CH₂CH₂CH₂S(═O)₂OH, —CH₂CH₂CH₂CH₂S(═O)₂OH, —CH₂CH₂CH₂CH₂CH₂S(═O)₂OH, —CH₂CH₂CH₂CH₂CH₂CH₂S(═O)₂OH, —CH(CH₃)OS(═O)₂OH, —CH₂CH(CH₃)OS(═O)₂OH, —CH₂CH₂CH(CH₃)OS(═O)₂OH, —CH₂CH₂CH₂CH(CH₃)OS(═O)₂OH, —CH₂CH₂CH₂CH₂CH(CH₃)OS(═O)₂OH, —CH₂CH₂CH₂CH₂CH₂CH(CH₃)OS(═O)₂OH, —CH₂OS(═O)₂OH, —CH₂CH₂OS(═O)₂OH, —CH₂CH₂CH₂OS(═O)₂OH, —CH₂CH₂CH₂CH₂OS(═O)₂OH, —CH₂CH₂CH₂CH₂CH₂OS(═O)₂OH, —CH₂CH₂CH₂CH₂CH₂CH₂OS(═O)₂OH, —C(═O)OH, —CH₂C(═O)OH, —CH₂CH₂C(═O)OH, —CH₂CH₂CH₂C(═O)OH, —CH₂CH₂CH₂CH₂C(═O)OH, or —CH₂CH₂CH₂CH₂CH₂C(═O)OH, or a salt or ion thereof. In some embodiments, the C₁₋₆ alkyl group is a C₃₋₆ alkyl group.

In some embodiments, the compound of formula (I) is:

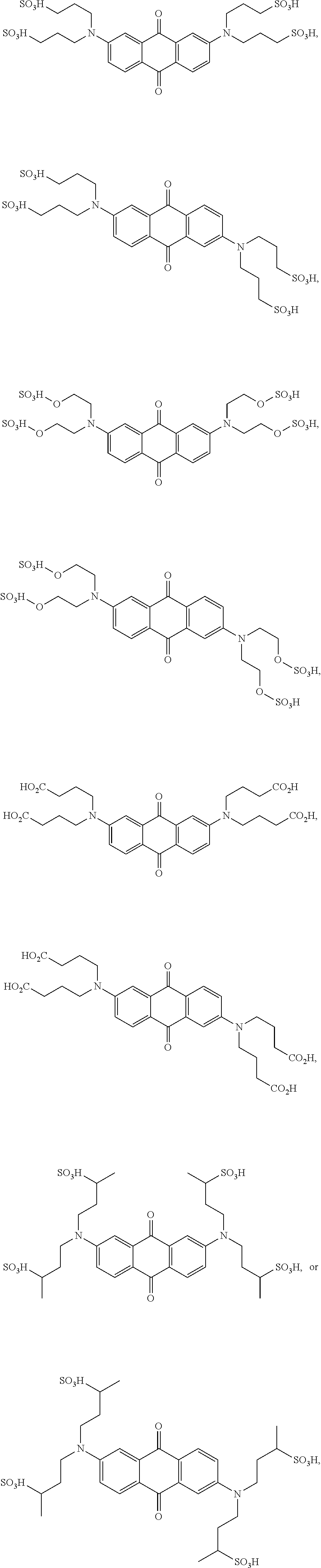

or an ion, salt, or hydroquinone thereof.

In some embodiments, the compound of formula (I) is produced by reacting 2,6-diaminoanthraquinone or 2,7-diaminoanthraquinone with 1,3-propanesultone, 1,3,2-dioxathiolane 2,2-dioxide, 3-methyl-1,2-oxathiolane 2,2-dioxide, or γ-butyrolactone. In certain embodiments, the compound of formula (I) is produced by reacting 2,6-diaminoanthraquinone or 2,7-diaminoanthraquinone with a mixture of at least two of 1,3-propanesultone, 1,3,2-dioxathiolane 2,2-dioxide, or 3-methyl-1,2-oxathiolane 2,2-dioxide.

In some embodiments, the compound of formula (I) is a salt including a lithium cation, a potassium cation, a cesium cation, or an organic cation, e.g., quaternary amine. In another aspect, the invention provides a method of producing a compound of formula (I) including reacting an aminoanthraquinone with 1,3-propanesultone, 1,3,2-dioxathiolane 2,2-dioxide, 3-methyl-1,2-oxathiolane 2,2-dioxide, γ-butyrolactone, or a combination thereof. In certain embodiments, the aminoanthraquinone is 2,6-diaminoanthraquinone or 2,7-diaminoanthraquinone.

In another aspect, the invention provides a battery including first and second electrodes, wherein the first electrode is in contact with a posolyte and/or the second electrode is in contact with a negolyte, wherein the posolyte and/or negolyte includes a compound of formula (I):

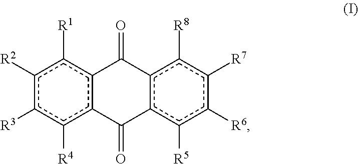

or an ion, salt, or hydroquinone thereof, where at least one of R¹, R², R³, R⁴, R⁵, R⁶, R⁷ and R⁸ is —N(R_{b})₂ or —N(R_{c})₂ where each R_{b} is independently an optionally substituted C₁₋₆ alkyl and each R_{c} is independently H or an optionally substituted C₁₋₆ alkyl, provided that when one R_{c} is H, the other R_{c} bound to the same N is an optionally substituted C₁₋₆ alkyl that is not CH₂CH₂C(═O)OH; and each remaining of R¹, R², R³, R⁴, R⁵, R⁶, R⁷ and R⁸ is independently selected from H; halo; optionally substituted C₁₋₆ alkyl; oxo; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —SRₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; and —P(═O)(ORₐ)₂; or any two adjacent groups selected from R⁸, R⁷, R⁶, and R⁵ are joined to form an optionally substituted 3-6 membered ring, where each Rₐ is independently H; optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group.

In some embodiments, the battery further comprises a positive and a negative redox active species, and the compound of formula (I) is a redox mediator. In some embodiments, the battery is a redox flow battery or a redox targeting battery.

In embodiments, the redox flow battery includes a redox active species which is or includes bromine, chlorine, iodine, molecular oxygen, vanadium, chromium, cobalt, iron, aluminum, manganese, cobalt, nickel, copper, or lead.

In another aspect, the invention provides a method of storing energy by oxidizing and/or reducing a compound of formula (I).

In certain embodiments, the compound of formula (I) is in a negolyte of a redox flow battery. In other embodiments, the compound of formula (I) is a redox mediator (e.g., in a redox targeting battery).

By “about” is meant ±10% of a recited value.

By “alkoxy” is meant a group of formula —OR, where R is an alkyl group, as defined herein.

By “alkyl” is meant straight chain or branched saturated groups from 1 to 6 carbons. Alkyl groups are exemplified by methyl, ethyl, n- and iso-propyl, n-, sec-, iso- and tert-butyl, neopentyl, and the like, and may be optionally substituted with one or more, substituents.

By “alkylene” is meant a divalent alkyl group.

By “alkyl thio” is meant —SR, where R is an alkyl group, as defined herein.

By “alkyl ester” is meant —COOR, where R is an alkyl group, as defined herein.

By “aryl” is meant an aromatic cyclic group in which the ring atoms are all carbon. Exemplary aryl groups include phenyl, naphthyl, and anthracenyl. Aryl groups may be optionally substituted with one or more substituents.

By “carbocyclyl” is meant a non-aromatic cyclic group in which the ring atoms are all carbon. Exemplary carbocyclyl groups include cyclopropyl, cyclobutyl, cyclopentyl, cyclohexyl, cycloheptyl, and cyclooctyl. Carbocyclyl groups may be optionally substituted with one or more substituents.

By “halo” is meant, fluoro, chloro, bromo, or iodo.

By “hydroxyl” is meant —OH. An exemplary ion of hydroxyl is —O—.

By “amino” is meant —NH₂. An exemplary ion of amino is —NH₃⁺.

By “nitro” is meant —NO₂.

By “carboxyl” is meant —COOH. An exemplary ion of carboxyl is —COO⁻.

By “phosphoryl” is meant —PO₃H₂. Exemplary ions of phosphoryl are —PO₃H⁻ and —PO₃²⁻.

By “phosphonyl” is meant —PO₃R₂, where each R is H or alkyl, provided at least one R is alkyl, as defined herein. An exemplary ion of phosphoryl is —PO₃R⁻.

By “oxo” is meant ═O.

By “sulfonyl” is meant —SO₃H. An exemplary ion of sulfonyl is —SO₃⁻.

By “thiol” is meant —SH.

By “heteroaryl” is meant an aromatic cyclic group in which the ring atoms include at least one carbon and at least one O, N, or S atom, provided that at least three ring atoms are present. Exemplary heteroaryl groups include oxazolyl, isoxazolyl, tetrazolyl, pyridyl, thienyl, furyl, pyrrolyl, imidazolyl, pyrimidinyl, thiazolyl, indolyl, quinolinyl, isoquinolinyl, benzofuryl, benzothienyl, pyrazolyl, pyrazinyl, pyridazinyl, isothiazolyl, benzimidazolyl, benzothiazolyl, benzoxazolyl, oxadiazolyl, thiadiazolyl, and triazolyl. Heteroaryl groups may be optionally substituted with one or more substituents.

By “heteroalkylene” is meant an alkylene group in which one or more CH₂ units are replaced with one or more heteroatoms selected from O, N, and S. Heteroalkylene groups can be substituted by oxo (═O). An exemplary heteroalkylene includes an amido group, e.g., —(CH₂)ₙC(O)NH(CH₂)ₘ—, wherein n and m are independently 1-6.

By “heterocyclyl” is meant a non-aromatic cyclic group in which the ring atoms include at least one carbon and at least one O, N, or S atom, provided that at least three ring atoms are present. Exemplary heterocyclyl groups include epoxide, thiiranyl, aziridinyl, azetidinyl, thietanyl, dioxetanyl, morpholinyl, thiomorpholinyl, piperazinyl, piperidinyl, pyrrolidinyl, tetrahydropyranyl, tetrahydrofuranyl, dihydrofuranyl, tetrahydrothienyl, dihydrothienyl, dihydroindolyl, tetrahydroquinolyl, tetrahydroisoquinolyl, pyranyl, pyrazolinyl, pyrazolidinyl, dihydropyranyl, tetrahydroquinolyl, imidazolinyl, imidazolidinyl, pyrrolinyl, oxazolidinyl, isoxazolidinyl, thiazolidinyl, isothiazolidinyl, dithiazolyl, and 1,3-dioxanyl. Heterocyclyl groups may be optionally substituted with one or more substituents.

By “hydrocarbyl” is meant a branched, unbranched, cyclic, or acyclic group including the elements C and H. Hydrocarbyl groups may be monovalent, e.g., alkyl, or divalent, e.g., alkylene. Hydrocarbyl groups may be substituted with groups including oxo (═O).

By an “oxygen protecting group” is meant those groups intended to protect an oxygen containing (e.g., phenol, hydroxyl, or carbonyl) group against undesirable reactions during synthetic procedures. Commonly used oxygen protecting groups are disclosed in Greene, “Protective Groups in Organic Synthesis,” 3rd Edition (John Wiley & Sons, New York, 1999), which is incorporated herein by reference. Exemplary oxygen protecting groups include acyl, aryloyl, or carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl; alkylcarbonyl groups, such as acyl, acetyl, propionyl, and pivaloyl; optionally substituted arylcarbonyl groups, such as benzoyl; silyl groups, such as trimethylsilyl (TMS), tert-butyldimethylsilyl (TBDMS), tri-iso-propylsilyloxymethyl (TOM), and triisopropylsilyl (TIPS); ether-forming groups with the hydroxyl, such methyl, methoxymethyl, tetrahydropyranyl, benzyl, p-methoxybenzyl, and trityl; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, and methyloxycarbonyl; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, allyloxycarbonyl, propargyloxycarbonyl, 2-butenoxycarbonyl, and 3-methyl-2-butenoxycarbonyl; haloalkoxycarbonyls, such as 2-chloroethoxycarbonyl, 2-chloroethoxycarbonyl, and 2,2,2-trichloroethoxycarbonyl; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl, and fluorenylmethyloxycarbonyl; and optionally substituted aryloxycarbonyl groups, such as phenoxycarbonyl, p-nitrophenoxycarbonyl, o-nitrophenoxycarbonyl, 2,4-dinitrophenoxycarbonyl, p-methyl-phenoxycarbonyl, m-methylphenoxycarbonyl, o-bromophenoxycarbonyl, 3,5-dimethylphenoxycarbonyl, p-chlorophenoxycarbonyl, and 2-chloro-4-nitrophenoxy-carbonyl); substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl); carbonyl-protecting groups (e.g., acetal and ketal groups, such as dimethyl acetal, and 1,3-dioxolane; acylal groups; and dithiane groups, such as 1,3-dithianes, and 1,3-dithiolane); carboxylic acid-protecting groups (e.g., ester groups, such as methyl ester, benzyl ester, t-butyl ester, and orthoesters; and oxazoline groups.

By a “nitrogen protecting group” is meant those groups intended to protect an amino group against undesirable reactions during synthetic procedures. Commonly used nitrogen protecting groups are disclosed in Greene, “Protective Groups in Organic Synthesis,” 3^{rd} Edition (John Wiley & Sons, New York, 1999), which is incorporated herein by reference. Nitrogen protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, alkaryl groups such as benzyl, triphenylmethyl, and benzyloxymethyl, and silyl groups, such as trimethylsilyl. Preferred nitrogen protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

Substituents may be optionally substituted with halo, optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —N(Rₐ)₂; —C(═O)Rₐ; —C(═O)ORₐ; —S(═O)₂Rₐ; —S(═O)₂ORₐ; —OS(═O)₂ORₐ, —P(═O)Rₐ₂; —O—P(═O)(ORₐ)₂, or —P(═O)(ORₐ)₂, or an ion thereof; where each Rₐ is independently H, optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group. Cyclic substituents may also be substituted with C₁₋₆ alkyl. In specific embodiments, substituents may be optionally substituted with halo, optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —NO₂; —ORₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; —O—P(═O)(ORₐ)₂, or —P(═O)(ORₐ)₂, or an ion thereof; where each Rₐ is independently H, optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group, and cyclic substituents may also be substituted with C₁₋₆ alkyl. In specific embodiments, alkyl groups may be optionally substituted with one, two, three, or, in the case of alkyl groups of two carbons or more, four substituents independently selected from the group consisting of halo, hydroxyl, C₁₋₆ alkoxy, SO₃H, SO₄H, amino, nitro, carboxyl, phosphoryl, phosphonyl, thiol, C₁₋₆ alkyl ester, optionally substituted C₁₋₆ alkyl thio, and oxo, or an ion thereof.

Exemplary ions of substituent groups are as follows: an exemplary ion of hydroxyl is —O⁻; an exemplary ion of —COOH is —COO⁻; exemplary ions of —PO₃H₂ are —PO₃H⁻ and —PO₃²⁻; an exemplary ion of —PO₃HRₐ is —PO₃Rₐ_{^{−}}, where Rₐ is not H; exemplary ions of —PO₄H₂ are —PO₄H⁻ and —PO₄²⁻; and an exemplary ion of —SO₃H is —SO₃_{^{−}}.

### BRIEF DESCRIPTION OF THE DRAWINGS

FIG. 1 shows the ¹H NMR spectrum of 2,6-N-TSAQ in D₂O-d6. The solvent peak at 4.7 pm was cut to increase other peaks. ¹H NMR (500 MHZ, D₂O) δ 7.65 (d, 2H), 7.00 (d, 2H), 6.76 (dd, 2H), 3.45 (t, 8H), 2.90 (t, 8H), 1.97 (m, 8H).

FIGS. 2(a)-2(b) show LC-MS traces of the synthesized 2,6-N-TSAQ. FIG. 2(a) shows the LC trace of 2,6-N-TSAQ; FIG. 2(b) shows the mass spectrum of the material eluted at 14.95 min in the LC trace. The peak at m/z=362.04 corresponds to the 2,6-N-TSAQ form with two protonated —SO₃_{^{−}} groups and two negative charges. Sample preparation: 0.1 M 2,6-N-TSAQ was diluted 100 times with HPLC water, and further diluted 100 times with acetonitrile/water co-solvents (volume ratio=1:1) to the desired concentration 10 μM. High-resolution LC-MS analysis was performed in the Small Molecule Mass Spectrometry Facility at Harvard University on a MiniLIMS. The elution solution was 0.1% v/v formic acid in acetonitrile. The ESI mass spectrum was recorded in negative ionization mode.

FIG. 3 shows the ¹H NMR spectrum of 2,6-O-DPSAQ in DMSO-d6. Solvent peaks are those not integrated. ¹H NMR (500 MHZ, DMSO-d6) δ 8.15 (d, 2H), 7.58 (d, 2H), 7.42 (dd, 2H), 4.30 (t, 4H), 2.59 (t, 4H), 2.07 (m, 4H).

FIG. 4 shows the ¹H NMR spectrum of 2,6-DPSAQ in DMSO-d6. Solvent peaks are those not integrated. ¹H NMR (500 MHZ, DMSO-d6) δ 8.12 (d, 2H), 7.99 (d, 2H), 7.74 (dd, 2H), 2.86 (t, 4H), 2.42 (t, 4H), 1.93 (m, 4H).

FIGS. 5(a)-5(c) show synthetic routes for three different anthraquinones: FIG. 5(a) 2,6-N-TSAQ; FIG. 5(b) 2,6-O-DPSAQ; and FIG. 5(c) 2,6-DPSAQ.

FIGS. 6(a)-6(b) show electrochemical and physical properties of three different anthraquinones: 2,6-DPSAQ, 2,6-N-TSAQ, and 2,6-O-DPSAQ. FIG. 6(a) shows cyclic voltammograms of 5 mM 2,6-N-TSAQ, 5 mM 2,6-DPSAQ in 1 M sodium chloride and 5 mM 2,6-O-TSAQ in 1 M lithium chloride with a scan rate of 100 mV/s. FIG. 6(b) shows water solubility comparison for 2,6-N-TSAQ, 2,6-O-DPSAQ and 2,6-DPSAQ.

FIG. 7 shows a Pourbaix diagram of 2,6-N-TSAQ.

FIGS. 8(a)-8(b) show polarization experiments of 2,6-N-TSAQ. FIG. 8(a) shows the open circuit voltage of 0.1 M 2,6-N-TSAQ/0.1 M potassium ferrocyanide+0.02 M potassium ferricyanide fuel cell at pH 14. FIG. 8(b) shows cell voltage and power density versus current density at room temperature at 10%, 30%, 50%, 70%, and 90% SOC. Cell configuration: 5 mL of 0.1 M 2,6-N-TSAQ pH 14|30 mL of 0.1 M K₄Fe(CN)₆, 0.02 M K₃Fe(CN)₆, pH 14. Nafion™ 212 was used as the ion-selective membrane between the AvCarb electrodes.

FIGS. 9(a)-9(d) show cell performance of a 0.1 M 2,6-N-TSAQ/ferrocyanide cell, with 2,6-N-TSAQ as the capacity limiting side. FIG. 9(a) shows the discharge capacity and Coulombic efficiency versus cycle time at pH 7 and 14. FIG. 9(b) shows the discharge capacity and Coulombic efficiency versus cycle number at pH 7 and 14. FIG. 9(c) shows the charge-discharge voltage profile of 2,6-N-TSAQ from selected cycles at pH 14 in FIG. 9(b). FIG. 9(d) shows the charge-discharge voltage profile of 2,6-N-TSAQ from selected cycles at pH 7 in FIG. 9(b).

FIG. 10 shows the energy efficiency and capacity contribution percentage at discharge voltage limit of 0.6 V versus cycle number of the 2,6-N-TSAQ/ferrocyanide cell in 1 M NaCl solution.

FIGS. 11(a)-11(c) show the ¹H NMR spectra (500 MHZ) of 2,6-N-TSAQ in D₂O solvent. FIG. 11(a) shows the initial 2,6-N-TSAQ spectrum. FIG. 11(b) shows the spectrum of a 0.1 M 2,6-N-TSAQ held at 65° C. for 8 days in 1 M sodium chloride. FIG. 11(c) shows the spectrum of a 0.1 M reduced state of 2,6-N-TSAQ held at 65° C. for 8 days in 1 M sodium chloride.

FIGS. 12(a)-12(b) shows thermodynamic analysis of anthraquinone disproportionation reaction. FIG. 12(a) is the disproportionation reaction of r-AQ at pH above its pKa2 and the corresponding two half reactions (omitting water molecules). FIG. 12(b) shows representative Pourbaix diagrams of anthraquinone, r-AQ, and anthrone. The pKa1 and pKa2 belong to r-AQ, and pKa (around 10) belongs to anthrone.

FIG. 13 shows cycling performance of 2,6-N-TSAQ/ferrocyanide flow batteries at pH 14 and 7 where 2,6-N-TSAQ is the capacity limiting side.

### DETAILED DESCRIPTION OF THE INVENTION

Redox flow batteries have emerged as promising systems for energy storage from intermittent renewable sources. The lifetime of these batteries is limited by electrolyte stability. The invention provides compounds that make electrolytes with high cycling stability that are inexpensive to produce.

The invention provides compounds (e.g., anthraquinones or anthrahydroquinones) of formula (I):

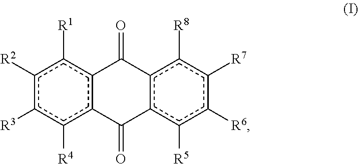

or an ion, salt, or hydroquinone thereof, where at least one of R¹, R², R³, R⁴, R⁵, R⁶, R⁷ and R⁸ is —N(R_{b})₂ or —N(R_{c})₂ wherein each R_{b} is independently an optionally substituted C₁₋₆ alkyl and each R_{c} is independently H or an optionally substituted C₁₋₆ alkyl, provided that when one R_{c} is H, the other R_{c} bound to the same N is an optionally substituted C₁₋₆ alkyl that is not —CH₂CH₂C(═O)OH; and each remaining of R¹, R², R³, R⁴, R⁵, R⁶, R⁷ and R⁸ is independently selected from H; halo; optionally substituted C₁₋₆ alkyl; oxo; optionally substituted C₃₋₁₀carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —SRₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; and —P(═O)(ORₐ)₂; or any two adjacent groups selected from R⁸, R⁷, R⁶, and R⁵ are joined to form an optionally substituted 3-6 membered ring, where each Rₐ is independently H; optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group. The dashed bonds indicate full conjugation.

In some embodiments, at least one R_{b} or R_{c} is an optionally substituted C₁₋₆ alkyl comprising a —S(═O)₂Rₐ; —S(═O)₂ORₐ; —OS(═O)₂ORₐ; —P(═O)Rₐ₂; or P(═O)(ORₐ)₂ substituent or is an optionally substituted C₂₋₆ alkyl including a —C(═O)ORₐ substituent.

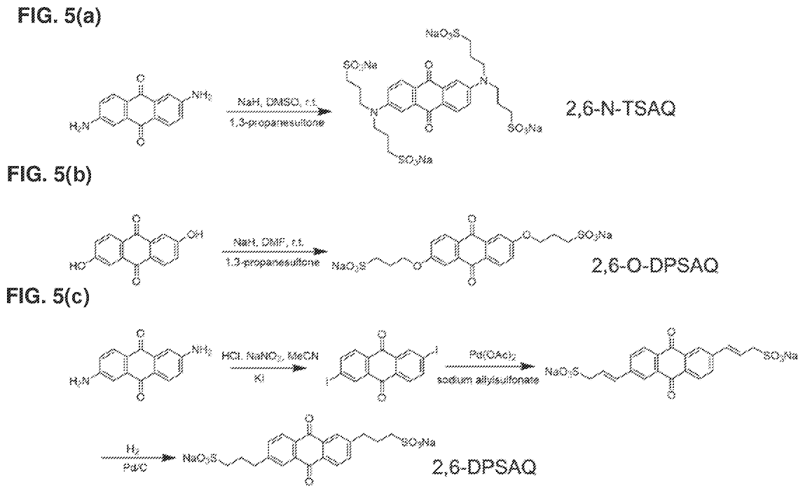

Compounds of formula (I) may be synthesized from diaminoanthraquinones (e.g., 2,6-diaminoanthraquinone (2,6-DAAQ)) and activated esters, e.g., cyclic activated esters, e.g., sultones (e.g., 1,3-propanesultone, 1,3,2-dioxathiolane 2,2-dioxide, or 3-methyl-1,2-oxathiolane 2,2-dioxide) or lactones (e.g., γ-butyrolactone). For example, 3,3′,3″,3′″-((9,10-anthraquinone-2,6-diyl)bis(azanetriyl))tetrakis(propane-1-sulfonate) (2,6-N-TSAQ) is synthesized from 2,6-DAAQ by a one-step N-allylation step at room temperature as shown in FIG. 5(a). Both raw materials 2,6-DAAQ and 1,3-propanesultone are inexpensive, thus, 2,6-N-TSAQ may be advantageously inexpensive at a mass-production scale.

### Flow Batteries

Flow batteries of the invention may include electrodes separated by electrolytes, e.g., a negolyte and a posolyte. Negolytes and posolytes contain redox active species which can store or transfer electrical energy by oxidation or reduction, e.g., at electrode surfaces. Compounds of formula (I) may be used in a negolyte as a redox active species. Where a compound of formula (I) is in a negolyte, other suitable redox active species may be used as a posolyte. Examples of redox active species for the posolyte include bromine, chlorine, iodine, molecular oxygen, vanadium, chromium, cobalt, iron (e.g., ferricyanide/ferrocyanide or a ferrocene derivative, e.g., as described in WO 2018/032003), aluminum, e.g., aluminum (III) biscitrate monocatecholate, manganese, cobalt, nickel, copper, or lead, e.g., a manganese oxide, a cobalt oxide, or a lead oxide. A benzoquinone may also be used as the redox active species. Other redox active species suitable for use in batteries of the invention are described in WO 2014/052682, WO 2015/048550, WO 2016/144909, and WO 2020/072406, the redox active species of which are incorporated by reference. The redox active species may be dissolved or suspended in solution (such as aqueous solution), be in the solid state, or be gaseous, e.g., molecular oxygen in air.

Compounds of formula (I) can also be used as a redox mediator for other aqueous redox batteries such as sulfur-based sodium (or potassium) polysulfides, or organosulfides or redox targeting batteries, e.g., having solid energy storage materials. In such batteries, a compound of formula (I) carries the charge between the electrode and an energy-storing substance, e.g., zinc, Mo₆S₈, LiTi₂(PO₄)₃, or NasV₂(PO₄)₃, or vice-versa. In such applications, long-term stability is much more important than high solubility or low mass-production cost, because the applications do not require high concentrations or large amounts of redox mediator.

In some embodiments, the electrolytes are both aqueous, where the negolyte and posolyte, e.g., an anthraquinone of formula (I) and redox active species, are in aqueous solution or aqueous suspension. In addition, the electrolyte may include other solutes, e.g., acids (e.g., HCl) or bases (e.g., LiOH, NH₄OH,

NaOH, or KOH) or alcohols (e.g., methyl, ethyl, or propyl) and other co-solvents to increase the solubility of a particular species, e.g., quinone/hydroquinone. Counter ions, such as cations, e.g., NH₄⁺, Li⁺, Na⁺, K⁺, Ce⁺, an organic cation, or a mixture thereof, may also be present. The battery may include a source of hydronium or hydroxide ions, e.g., an acid or base, to, e.g., control the pH of the negolyte and/or posolyte. In certain embodiments, the pH of the posolyte and/or negolyte may be >7, e.g., at least 8, 9, 10, 11, 12, 13, or 14, 8-14, 9-14, 10-14, 11-14, 12-14, 13-14, or about 14. In certain embodiments, the pH of the posolyte and/or negolyte may be <7, e.g., less than 7, 6, 5, 4, 3, 2, 1, or 0, e.g., 7-1, 7-6, 6-4, 5-3, 4-2, 3-1, or 1-0. The electrolytes may or may not be buffered to maintain a specified pH. The negolyte and posolyte will be present in amounts suitable to operate the battery, for example, from 0.01-15 M,, e.g., about 0.01-1M (e.g., about 0.01-0.05M, 0.05-0.1M, 0.1-0.5M, or about 0.5-1M, e.g., about 0.01M, 0.05M, 0.1M, 0.15M, 0.2M, 0.25M, 0.5M, or 1M), or, e.g., about 1-15 M (e.g., about 1-4 M, 2-6M, 3-7, M, 5-8M, 3-9M, 5-10M, 7-15M, 8-12M, 10-13M, or about 12-15M, e.g., about 1M, 2M, 5M, 10M, 12M, or 15M). In some embodiments, the solution is at least 10%, 20%, 30%, 40%, 50%, 60%, 70%, or 80% water, by mass. Negolytes, e.g., quinones, hydroquinones, salts, and/or ions thereof may be present in a mixture.

In addition to water, solutions or suspensions may include alcohols (e.g., methyl, ethyl, or propyl alcohol) and other co-solvents to increase the solubility of a particular species. In some embodiments, the solution or suspension is at least 10%, 20%, 30%, 40%, 50%, 60%, 70%, or 80% water, by mass. Alcohol or other co-solvents may be present in an amount required to result in a particular concentration of species. The pH of the aqueous solution or suspension may also be adjusted by addition of acid or base, e.g., to aid in solubilizing a species.

Electrodes suitable for use with negolytes of the invention include any carbon electrode, e.g., glassy carbon electrodes, carbon paper electrodes, carbon felt electrodes, or carbon nanotube electrodes. Other suitable electrodes may include metals such as stainless steel, copper, bismuth, or lead. Titanium electrodes may also be employed. Electrodes can also be made of a high specific surface area conducting material, such as a nanoporous metal sponge (T. Wada, A. D. Setyawan, K. Yubuta, and H. Kato, Scripta Materialia 65, 532 (2011)), which has been synthesized previously by electrochemical dealloying (J. D. Erlebacher, M. J. Aziz, A. Karma, N. Dmitrov, and K. Sieradzki, Nature 410, 450 (2001)), or a conducting metal oxide, which has been synthesized by wet chemical methods (B. T. Huskinson, J. S. Rugolo, S. K. Mondal, and M. J. Aziz, arXiv:1206.2883 [cond-mat.mtrl-sci]; Energy & Environmental Science 5, 8690 (2012); S. K. Mondal, J. S. Rugolo, and M. J. Aziz, Mater. Res. Soc. Symp. Proc. 1311, GG10.9 (2010)). Chemical vapor deposition can be used for conformal coatings of complex 3D electrode geometries by ultra-thin electrocatalyst or protective films. Electrodes suitable for other redox active species are known in the art.

Batteries of the invention may include a barrier that separates the electrodes. The barrier allows the passage of ions, such as sodium or potassium, but not a significant amount of the negolyte or other redox active species. Examples of ion conducting barriers are NAFION®, i.e., sulfonated tetrafluoroethylene based fluoropolymer-copolymer, FUMASEP®, i.e., non-fluorinated, sulfonated polyaryletherketone-copolymer, e.g., FUMASEP® E-620 (K), hydrocarbons, e.g., polyethylene, and size exclusion barriers, e.g., ultrafiltration or dialysis membranes with a molecular weight cut off of 100, 250, 500, or 1,000 Da. For size exclusion membranes, the required molecular weight cut off is determined based on the molecular weight of the negolytes and posolytes employed. Porous physical barriers may also be included, e.g., when the passage of redox active species is tolerable.

A battery of the invention may include additional components as is known in the art. Negolytes and posolytes may be housed in a suitable reservoir. A battery may further include one or more pumps to pump aqueous solutions or suspensions past one or both electrodes. Alternatively, the electrodes may be placed in a reservoir that is stirred or in which the solution or suspension is recirculated by any other method, e.g., convection, sonication, etc. Batteries may also include graphite flow plates and corrosion-resistant metal current collectors.

The balance of the system around the cell includes fluid handling and storage, and voltage and round-trip energy efficiency measurements can be made. Systems configured for measurement of negolyte and posolyte flows and pH, pressure, temperature, current density and cell voltage may be included and used to evaluate cells. Fluid sample ports can be provided to permit sampling of both electrolytes, which will allow for the evaluation of parasitic losses due to reactant crossover or side reactions. Electrolytes can be sampled and analyzed with standard techniques.

Suitable cells, electrodes, membranes, and pumps for redox flow batteries are known in the art, e.g., WO 2014/052682, WO 2015/048550, WO 2016/144909, and WO 2020/072406, the battery components of which are hereby incorporated by reference.

### Methods

### Synthesis

The invention provides a method of producing compounds of formula (I) by reacting an aminoanthraquinone with 1,3-propanesultone, 1,3,2-dioxathiolane 2,2-dioxide, 3-methyl-1,2-oxathiolane 2,2-dioxide, γ-butyrolactone, or a combination thereof. Compounds of formula (I) may be synthesized via a one-step nucleophilic reaction of activated esters with aminoanthraquinones. For example, compounds of formula (I) may be synthesized from 2,6-diaminoanthraquinone (2,6-DAAQ) and sultones (e.g., 1,3-propanesultone, 1,3,2-dioxathiolane 2,2-dioxide, or 3-methyl-1,2-oxathiolane 2,2-dioxide) or lactones (e.g., γ-butyrolactone). The nucleophilic addition reaction may first involve deprotonating the diaminoanthraquinone (e.g., with sodium hydride, e.g., in anhydrous dimethyl sulfoxide or N,N-Dimethylformamide). With high reactivity activated esters, e.g., 1,3-propanesultone, the reaction can occur readily at room temperature with high purity and yield, making these particularly suitable for mass production.

### Formula (I) in Energy Storage Application

Compounds of formula (I) may be used in energy storage applications, e.g., in redox flow batteries. Typically, the compound of formula (I) is in the negolyte and therefore in its reduced, hydroquinone form when fully charged. To charge a battery of the invention, a voltage is applied across the electrodes. Applying a voltage across the electrodes causes redox active species in the posolyte and negolyte (e.g., a compound of formula (I)) to be, respectively, oxidized and reduced (e.g., reducing the compound of formula (I) from its quinone form to its hydroquinone form). The oxidized and reduced redox active species may be pumped to reservoirs for storage and to allow more redox active species to be oxidized and reduced at the electrodes. A battery of the invention is discharged by connecting a load across the electrodes. As the battery is discharged, the redox active species in the posolyte and negolyte are, respectively, reduced and oxidized (e.g., when the compound of formula (I) is in the negolyte, it is oxidized from its hydroquinone form to its corresponding quinone form).

In other batteries of the invention, the compounds of formula (I) may act as charge transfer mediator in the reduction or oxidation of a secondary species. For example, in a redox targeting battery, a compound of formula (I) is oxidized and reduced at the electrodes and then transfers the electrical energy to a solid redox active species by being reduced or oxidized by electron transfer reactions at surface of the solid redox active materials.

### EXAMPLES

The invention will be further described by the following non-limiting example.

### Example 1

Here, we report inexpensive and low redox-potential anthraquinone with outstanding cycling stability. The anthraquinone sodium 3,3′,3″,3′″-((9,10-anthraquinone-2,6-diyl)bis(azanetriyl))tetrakis(propane-1-sulfonate) (2,6-N-TSAQ) was synthesized from 2,6-diaminoanthraquinone (2,6-DAAQ) via a one-step N-alkylation route. The reduction potential of 2,6-N-TSAQ at pH 12 and above is −0.62 V vs. SHE, which is 120 mV lower than the oxygen-linked anthraquinone sodium 3,3′-((9,10-anthraquinone-2,6-diyl)bis(oxy))bis(propane-1-sulfonate) (2,6-O-DPSAQ) and 170 mV lower than the carbon-linked anthraquinone sodium 3,3′-(9,10-anthraquinone-2,6-diyl)bis(propane-1-sulfonate) 2,6-DPSAQ. Pairing with ferri/ferrocyanide, it forms a 1.14 V full cell and shows a maximum peak power density of 0.18 W/cm² at pH 14. The capacity fade rate of 2,6-N-TSAQ is 0.025%/day at pH 14, making it one of the most stable redox organic molecules ever reported, and the first highly stable anthraquinone with a redox potential below −0.6 V vs. SHE. In contrast, the capacity fade rate at neutral condition (1 M sodium chloride) is as high as 2.6%/day. The substantial difference in anthraquinone cycling stability at different pH values is due to their differences in Gibbs free energy change for the anthrone formation reaction. These results provide guidance to improve the cell performance of anthraquinone-based negolyte and highlight the great potential of organic synthesis towards inexpensive and stable electrolytes for grid-scale energy storage application.

### Experimental Section

Chemicals: 2,6-diaminoanthraquione (97%), 1,3-propanesultone (98%), sodium hydride (60% in mineral oil), anhydrous dimethyl sulfoxide, anhydrous N,N-Dimethylformamide, potassium carbonate, and palladium (II) acetate (98%) were purchased from Sigma-Aldrich. 2,6-dihydroxyanthraquinone (98%) was purchased from AK scientific. Sodium allylsulfonate (94%) was purchased from Ambeed, inc. Ltd. Hydrogen gas was purchased from Airgas. The materials were directly used without further purification.

### Synthesis of 2,6-N-TSAQ

3 g of 2,6-diaminoanthraquinone (12.59 mmol) was added to 50 mL anhydrous dimethyl sulfoxide. Then 2.1 g sodium hydride (60%, 52.46 mmol) was added to the solution under vigorous stirring. After 15 minutes, 6.41 g 1,3-propanesultone (98%, 52.46 mmol) was added to the above mixture. The solution was stirred at room temperature for 1 hour. Afterward, ethyl acetate was added to the solution to collect the red solid. The crude product was washed with ethyl acetate to remove any mineral oil. Yield: 9.7 g (95%).

### Synthesis of 2,6-DPSAQ

2,6-diiodoanthraquinone was synthesized according to methods known in the art. A mixture of 2 g 2,6-diiodoanthraquinone (4.35 mmol), 0.75 g sodium allylsulfonate (5.22 mmol), 0.72 g potassium carbonate (5.22 mmol) and 49 mg palladium acetate (0.22 mmol) was heated in 40 ml water in a pressure vessel at 120° C. for overnight. The mixture solution was filtered to remove any insoluble gradients. The filtrate collected and added to a 20 mL methanol solution. The solution was stirred in a hydrogen atmosphere overnight, then the solution evaporated with vacuum and the resulting solid collected. Yield: 1.51 g (70%).

### Full Cell Measurements

Flow battery experiments were performed with cell and hardware from Fuel Cell Tech. (Albuquerque, NM). Pyrosealed POCO graphite flow plates with serpentine flow designs were used for both electrodes. Each electrode comprised a single 5 cm² geometric surface area sheet of AvCarb carbon electrode. For 2,6-N-TSAQ/ferrocyanide full cell tests, a Nafion™ 212 membrane was used to serve as the ion-selective membrane. The Nafion membrane was soaked in the supporting electrolyte (sodium hydroxide or sodium chloride) for at least 24 hours before use. Viton sheets were used to cover the outer portion space between the electrodes. Torque used for cell assembly was 60 lb-in (6.78 Nm) on each of eight 1/4-28 bolts. The electrolytes were fed into the cell through fluorinated ethylene propylene (FEP) tubing at a rate of 60 mL/min, controlled by Cole-Parmer 6 Masterflex L/S peristaltic pumps. The cell was run inside a glove box (1 ppm O₂). Cell polarization measurements and charge-discharge cycling were conducted using a Biologic BCS-815 battery cycler. Long-term cycling of the 0.1 M 2,6-N-TSAQ/ferrocyanide cell was achieved at +40 mA cm⁻² with potential holds at 1.4 V for charging and 0.6 V for discharging until the current density dropped to 2 mA cm⁻². The polarization curves were obtained by charging to a desired state of charge first and then polarizing via linear sweep voltammetry at a rate of 100 mV s⁻¹.

A glassy carbon (BASi, 3 mm diameter) working electrode, an Ag/AgCl reference electrode (BASi, 3 M NaCl solution), and a graphite counter electrode were used in the three-electrode system for all CV tests. The scan rates for CV tests were 10, 20, 50, 100, and 200 mV s⁻¹. The diffusion coefficient D was calculated based on the Randles-Sevcik equation: Ip=269000×n^{1.5} AD^{0.5}v^{0.5}C, where Ip is the peak current in amps, n is the number of electrons involved (n=2 for 2,6-N-TSAQ) in the redox reaction, A is the active surface area (0.0707 cm²), D is the diffusion coefficient in cm²/s, v is the scan rate in V/s, and C is the concentration of redox species in mol/cm³.

### Results and Discussion

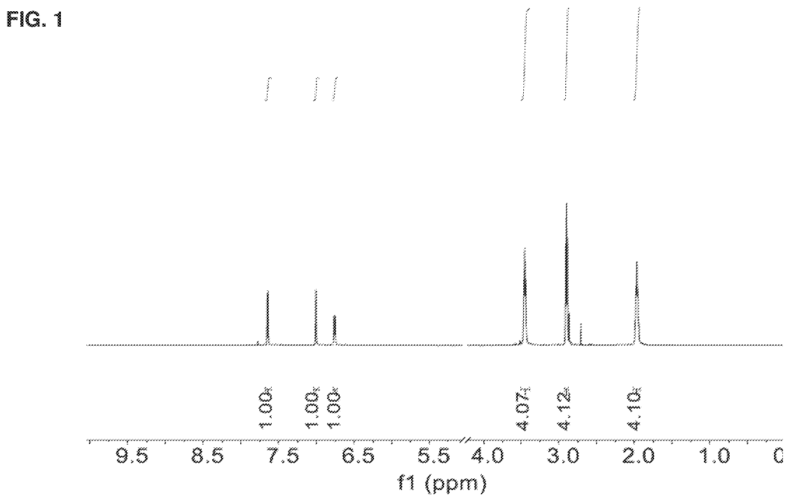

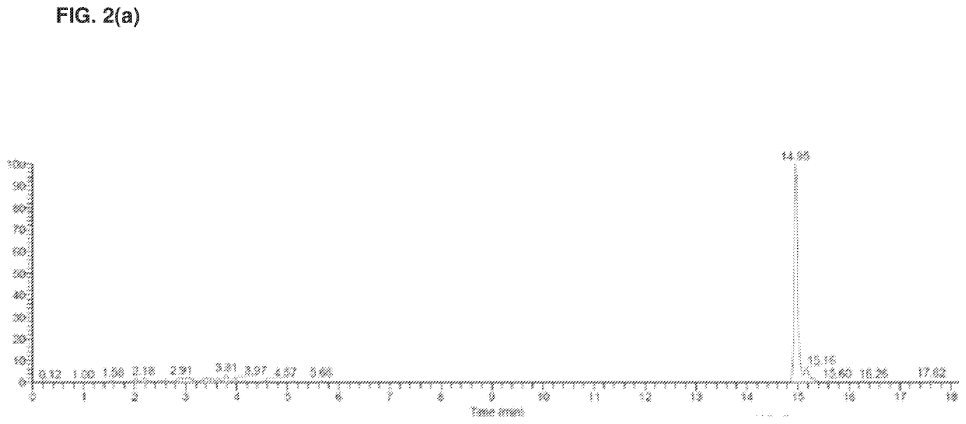

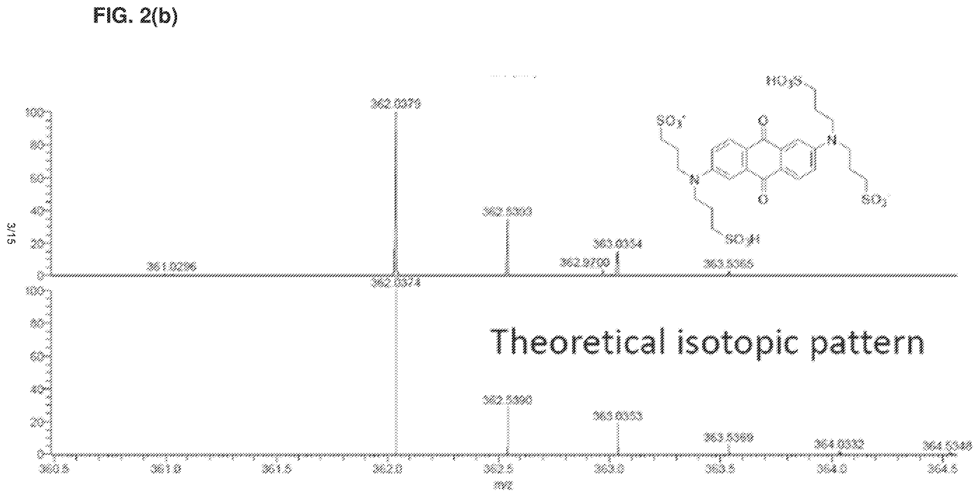

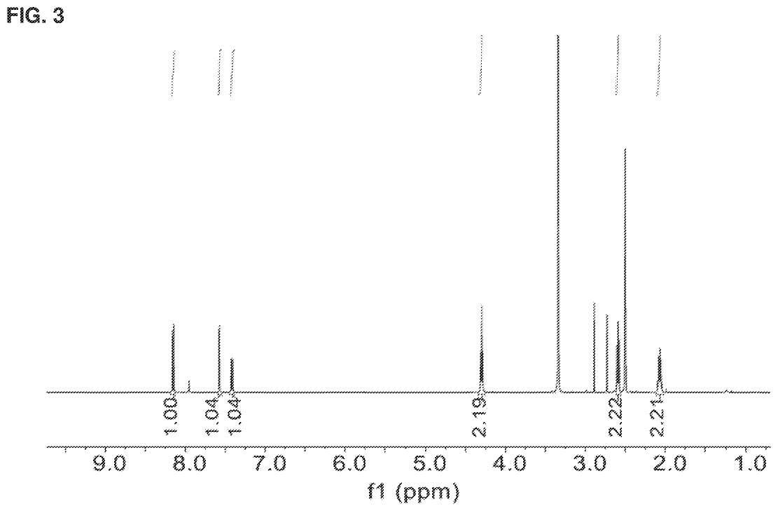

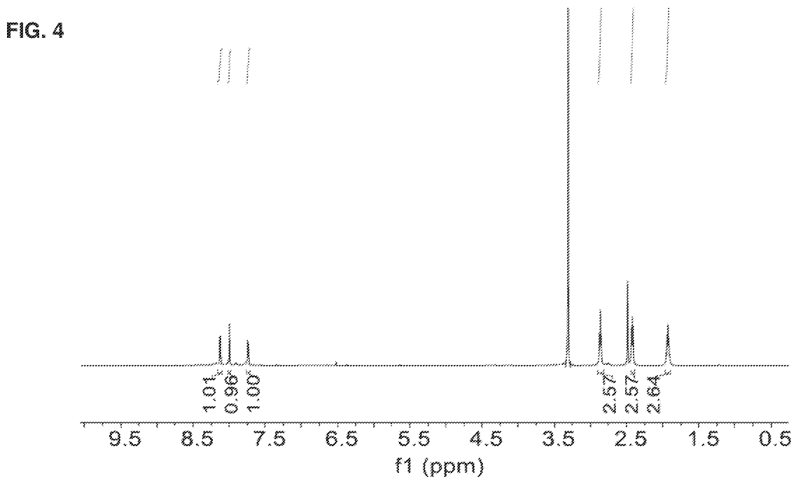

FIG. 5 illustrates the synthetic routes for three different anthraquinones 2,6-N-TSAQ, 2,6-O-DPSAQ and 2,6-DPSAQ. The structure of 2,6-N-TSAQ was verified by ¹H nuclear magnetic resonance (NMR) and high-resolution liquid chromatography-mass spectrometry, as shown in FIG. 1 and FIG. 2. The structures of 2,6-O-DPSAQ and 2,6-DPSAQ were verified by ¹H NMR, as shown in FIG. 3 and FIG. 4. 2,6-N-TSAQ and 2,6-O-DPSAQ were synthesized via one-step nucleophilic reaction. 2,6-N-TSAQ was produced from 2,6-DAAQ, and 2,6-O-DPSAQ was synthesized from 2,6-dihydroxyanthraquinone (2,6-DHAQ). In both cases, sodium hydride was used to fully deprotonate the anthraquinone precursors in anhydrous dimethyl sulfoxide or N,N-Dimethylformamide. Afterward, the deprotonated anthraquinone was reacted with 1,3-propanesultone at room temperature for 1 hour to afford 2,6-N-TSAQ or 2,6-O-DPSAQ. Benefiting from the high reactivity of 1,3-propanesultone, the reaction readily occurs at room temperature with high purity and yield, making it very suitable for mass production.

2,6-DPSAQ was synthesized from 2,6-DAAQ with three steps. First, 2,6-DAAQ was iodized to form 2,6-diiodoanthraquinone. Subsequently, it reacted with sodium allylsulfonate via Heck reaction followed by a hydrogenation step to yield 2,6-DPSAQ. The three-step reaction involving precious metal catalysts makes it less attractive compared with the one-step synthesis of 2,6-N-TSAQ and 2,6-O-DPSAQ. Since the laboratory cost of precursor 2,6-DAAQ is much lower than that of 2,6-DHAQ, 2,6-N-TSAQ could be the most inexpensive anthraquinone among the three at mass production scale, decreasing the capital cost of AORFBs.

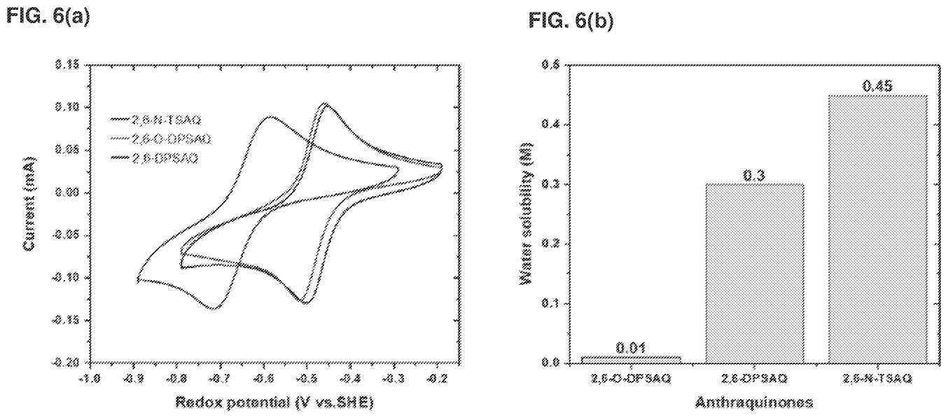

FIG. 6 (a) shows the cyclic voltammograms (CV) of 2,6-DPSAQ, 2,6-O-DPSAQ, and 2,6-N-TSAQ. The reduction potential of 2,6-N-TSAQ is-0.62 V vs. SHE in 1M NaCl, which is 120 mV and 170 mV lower than that of 2,6-O-DPSAQ and 2,6-DPSAQ in 1M NaCl, respectively. The low redox potential of 2,6-N-TSAQ is attributed to the strong electron donating effect of N-alkyl group. It contributes to form a high working voltage and high power density flow battery. The water solubilities of 2,6-DPSAQ, 2,6-O-DPSAQ, and 2,6-N-TSAQ are shown in FIG. 6 (b). Surprisingly, 2,6-O-DPSAQ exhibited an extremely low solubility of 10 mM in deionized water and 1 M lithium chloride, and an even lower solubility of less than 5mM in 1 M sodium chloride. The water solubility of 2,6-DPSAQ and 2,6-N-TSAQ are 0.3 M and 0.45 M, respectively. The solubility of 2,6-N-TSAQ could be boosted to 0.65 M with ammonium ion exchange. The ammonium cation, however, sacrifices the redox potential of 2,6-N-TSAQ as the local pH is always lower than the pKa2 of 9,10-dihydroxyanthracene, which is also observed in the reported 9,10-anthraquinone-2,7-disulfonic diammonium salt.

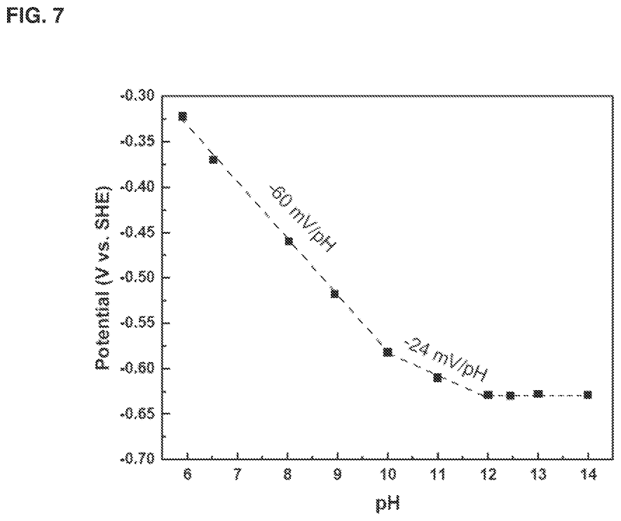

Given the higher water solubility, lower redox potential and possible lower synthetic cost, 2,6-N-TSAQ was selected for further electrochemical study. One advantage of 2,6-N-TSAQ over other low redox potential anthraquinones is that it has four negative charges, thus the intramolecular Coulomb repulsion is very large so that its collision factor is low. Based on Marcus theory, the disproportionation reaction that is known to cause the capacity decay in anthraquinone species is suppressed. The multiple negative charges also decrease the molecular permeability across the cation exchange membrane, which can also increase the cell lifetime. The Pourbaix diagram of 2,6-N-TSAQ, as shown in FIG. 7, indicates the molecule undergoes a two-proton/two-electron process below pH 10, a one-proton/two-electron process between pH 10-12, and it becomes pH independent at high pH (pH>12) with a redox potential around −0.62 V vs. SHE. The pH in the Pourbaix diagram represents the local pH of anthraquinone molecules. In an unbuffered case, e.g., 1 M NaCl, though the initial pH is 7, reduction consumes two protons per anthraquinone molecule, immediately increasing the local pH, exhibiting a formal potential −0.62 V for 2,6-N-TSAQ which is equal to the redox potential in the high pH region. Based on the Pourbaix diagram, the pKa1 and pKa2 of reduced 2,6-N-TSAQ are calculated to be around 10 and 12, respectively, which are slightly higher than those of high redox potential anthraquinones. This is attributed to the strong electron donating effect of N-alkyl group decreasing the acidity of the hydroxy groups of the 9,10-dihydroxyanthracene (reduced state of anthraquinone).

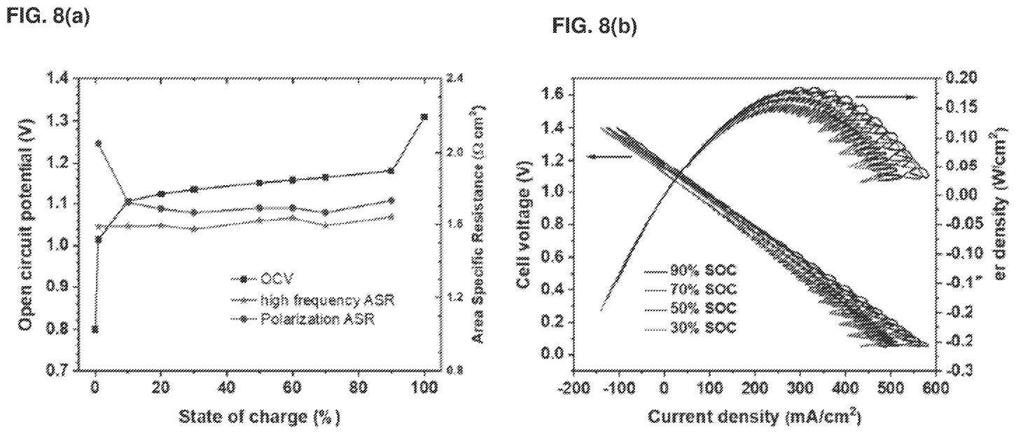

Polarization experiments of a 0.1 M 2,6-N-TSAQ/ferrocyanide full cell at pH 14 were performed at various states of charge. The negolyte comprised 5 mL of 0.1 M 2,6-N-TSAQ at pH 14 and posolyte contained 30 mL of 0.1 M potassium ferrocyanide and 0.02 M potassium ferricyanide at pH 14 to ensure that the negolyte was always the capacity limiting side. The cell was constructed from graphite flow plates and AvCarb carbon electrodes, separated by a Nafion 212 membrane pretreated in 1 M KOH. To access the full capacity, the cell was charged and discharged to 1.4 V and 0.6 V, respectively, with a potential hold until the current dropped to 2 mA/cm². The open-circuit voltage (OCV) increases from 0.8 V to 1.31 V as the state of charge (SOC) increases from ˜0% to ˜100% (FIG. 8 (a)). The OCV at 1%, 20%, 50% and 90% SOC are around 1.02 V, 1.11 V, 1.14 V and 1.18 V, respectively. The OCV increase of 0.2 V from ˜0 to 1% SOC, and of only 0.08 V from 10% to 90% SOC, indicate the full utilization of 2,6-N-TSAQ under the operating conditions and that the redox potential of 2,6-N-TSAQ is a constant during cycling, in qualitative consistency with the Nernst equation. The OCV at 50% SOC is similar to the cell voltage expected from the CV result. The peak galvanic power density at 10% SOC was 0.15 W cm⁻², increasing to 0.18 W cm² at 90% SOC (FIG. 8 (b)), which is higher than that of many other redox organics. The power density is mainly limited by the high-frequency area-specific resistance, which is dominated by the membrane resistance (FIG. 8 (a)) with a value around 1.6 Ω·cm². Therefore, the power density is expected to be improved with a lower resistance membrane.

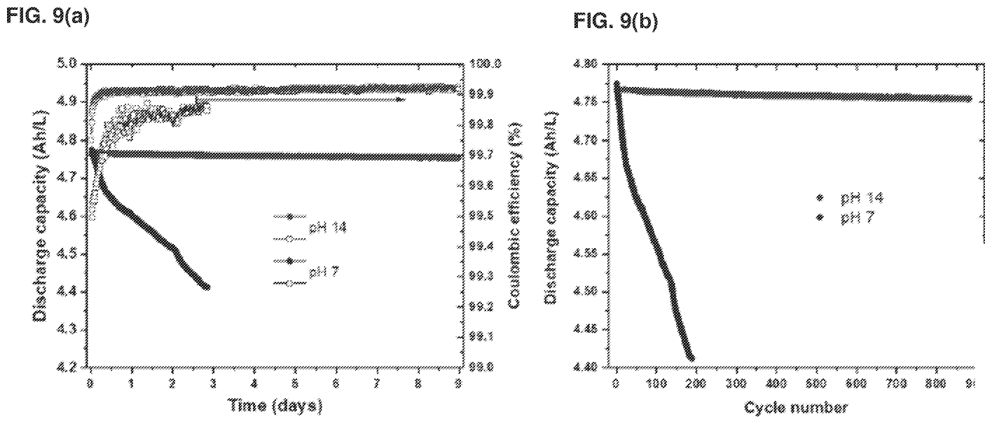

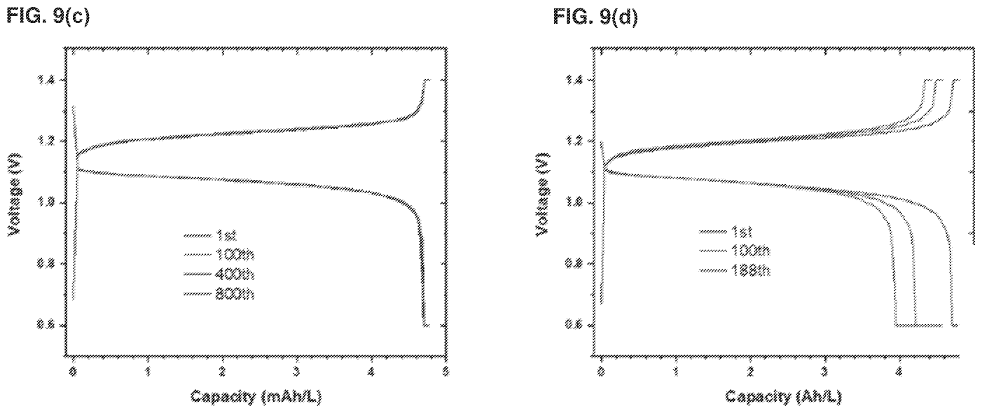

Long-term cycling testing of 0.1 M 2,6-N-TSAQ/ferrocyanide was performed with the same cell. The cell was cycled at 40 mA cm² with potential holds at 1.4 V for charging and 0.6 V for discharging until the current density dropped to 2 mA cm⁻². The initial discharge capacity was 4.764 Ah L⁻¹, corresponding to a capacity utilization of 88.9% of the theoretical value. However, the OCV at different SOC values in FIG. 8 (a) and the typical voltage profile in FIG. 9 (c) indicates it accesses the complete capacity of 2,6-N-TSAQ under such conditions. The differences between the actual capacity and the theoretical value could come from non-redox active impurities such as water or salts in the sample. After 9 days of full SOC range cycling, the discharge capacity decreased to 4.754 Ah/L, corresponding to a temporal capacity fade rate of 0.025%/day or a cycle-denominated capacity fade rate of 0.00024%/cycle. The average coulombic efficiency was determined to be above 99.9%. The voltage vs. capacity profile at different cycles in FIG. 4 (c) are almost invariant, indicating the ultra-stable cell performance of 2,6-N-TSAQ at pH 14. These results stand out as among the most stable electrolytes for AORFBs ever reported.

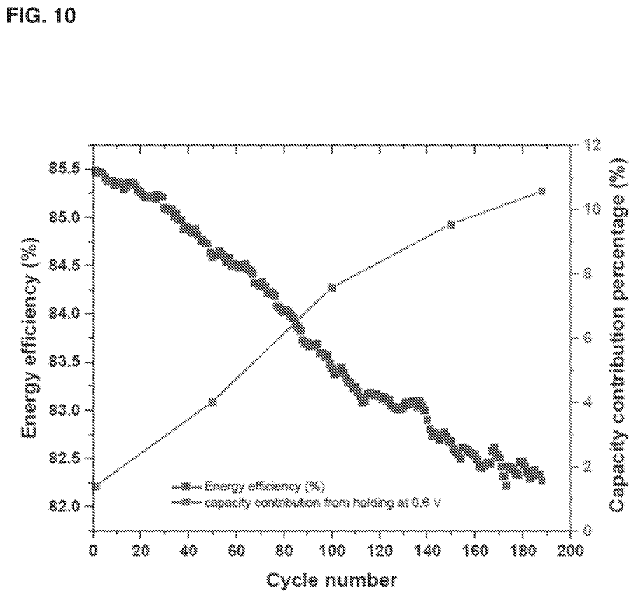

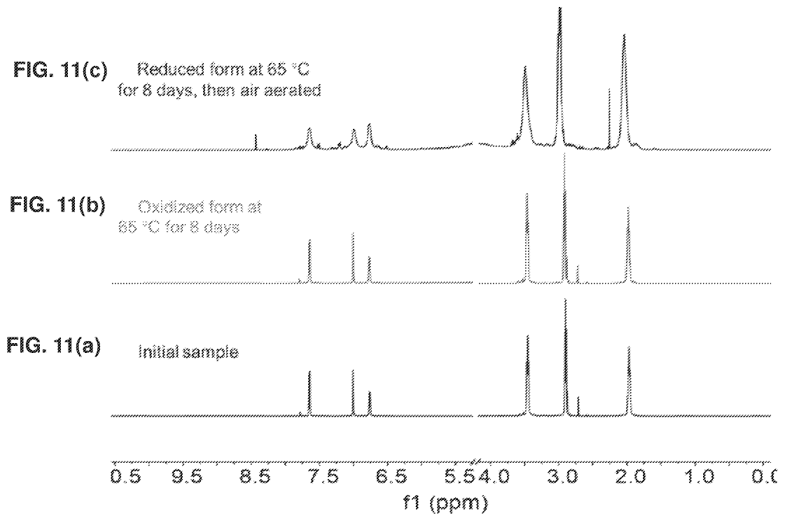

For comparison, the long-term cycling of the 0.1 M 2,6-N-TSAQ/ferrocyanide cell was operated at neutral condition (1 M sodium chloride) but, otherwise, the same conditions. The initial volumetric capacity was 4.774 Ah L⁻¹, which is similar to that at pH 14. After 2.84 days cycling, the discharge capacity dropped to 4.412 Ah/L, corresponding to a capacity fade rate of 2.6%/day, which is around 2 orders magnitude higher than that at pH 14. And the coulombic efficiency is around 99.8% over the whole cycling process, which is slightly lower than that at pH 14. Furthermore, the discharge capacity contribution from potential at the discharge voltage limit of 0.6 V increases, and energy efficiency decreases, with cycling, as shown in FIG. 10, further illustrating the instability of 2,6-N-TSAQ at neutral condition. To investigate the origin of instability of 2,6-N-TSAQ at neutral condition, both oxidized and reduced forms of 2,6-N-TSAQ were stored in 1 M NaCl at 65° C. for 8 days. As shown in FIG. 11, no apparent decomposition was found in the ¹H NMR of the oxidized state, indicating the excellent stability of 2,6-N-TSAQ in its oxidize state. In contrast, the reduced sample 2,6-N-TSAQ shows a large quantity of decomposition after 8 days treatment. Our working hypothesis to interpret these results is that the reduced 2,6-N-TSAQ undergoes the disproportionation reaction that is known in anthraquinone.

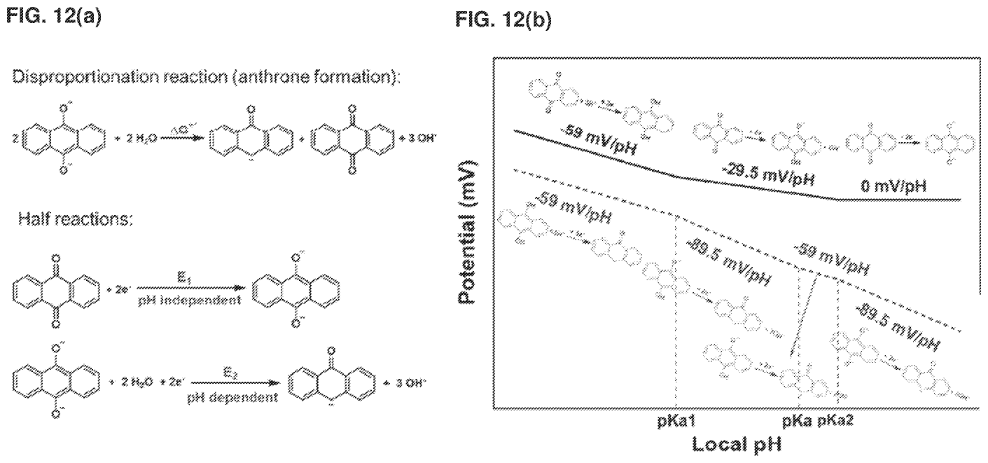

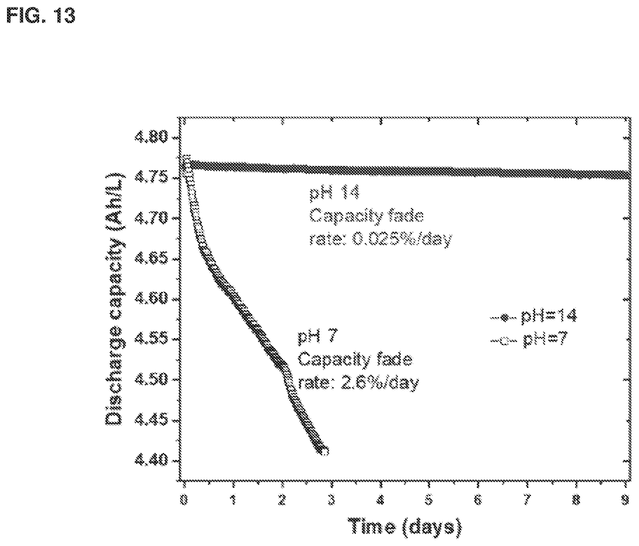

The substantial difference in the stability of reduced anthraquinone at pH 7 and 14 could be explained by the following thermodynamic arguments. As shown in FIG. 12, the disproportionation reaction of reduced anthraquinone (r-AQ) was intrinsically the overall reaction of the two half reactions: anthraquinone/r-AQ and r-AQ/anthrone. The Gibbs free energy change ΔG^{o}′ at a quasi-standard condition could be expressed as ΔG^{o}′=−2×F×(E2−E1), quasi standard condition represents a state that similar to the standard condition except the pH is fixed at a certain value, and E1 and E2 are the electrode redox potential of the two half-reactions at quasi standard condition. The Pourbaix diagram of anthraquinone, r-AQ and anthrone are shown in FIG. 12. The redox reaction of anthraquinone/r-AQ and r-AQ/anthrone are proton-coupled electron transfer reaction. When the local pH<pKa1 of r-AQ, both redox pairs anthraquinone/r-AQ and r-AQ/anthrone undergo a two-proton/two-electron process. When the local pH is between pKa1 of r-AQ and pKa of anthrone, anthraquinone/r-AQ undergoes one-proton/two-electron process, and r-AQ/anthrone undergoes three-proton/two-electron reaction. When the local pH increased to pKa˜pKa2 region, anthraquinone/r-AQ still experiences one-proton/two-electron process, while r-AQ/anthrone undertakes two-proton/two-electron process. When pH is above pKa2 of r-AQ, the redox reaction of anthraquinone/r-AQ becomes pH independent, and r-AQ/anthrone undergoes three-proton/two-electron reaction. The Gibbs free energy difference of one molar anthrone formation at pH 14and 12, as shown in FIG. 12(b) is around ΔG=−2×96485 C/mol×(−89.5 mV/pH×2 pH)×1 V/(1000 mV)=34.54 kJ/mol.

Similarly, the Gibbs free energy difference of one molar anthrone formation at pH 14 and 10 could be as large as 46.13˜57.70 kJ/mol depending on the pKa1, pKa and pKa2 values, indicating that anthrone formation is greatly suppressed at pH 14 than that at lower pH values. Consequently, anthraquinone-based flow batteries for which anthrone formation is the dominant loss mechanism exhibit much better cycling performance at high pH than that at a lower pH. Furthermore, the Gibbs free energy change for anthrone formation reaction at a 100% SOC state (no anthrone, no anthraquinone, and 100% 9,10-dihydroxyanthracene) is always negative, indicating that for any given anthraquinone, the disproportionation reaction at full SOC state is always thermodynamically favorable. Additionally, the redox potential E1 for anthraquinone reduction at quasi standard condition is equal to that of anthraquinone reduction at 50% SOC cycling. When the state of charge of anthraquinone increases from 90% to 99%, the ΔG for anthrone formation decreases around 8.314 J/mol/K 298.15 K (In 0.1/0.9²− 0.01/0.99²)=6.18 kJ/mol if assuming the change of anthrone concentration is negligible. And if the state of charge of anthraquinone cycling increases from 90% to 99.9%, the Gibbs free energy change for anthrone formation decreases around 11.93 kJ/mol. Therefore, to suppress anthrone formation, charging to high SOC should be avoided, e.g., it is desired not to conduct a potential hold at high potential. However, to accurately measure a capacity fade rate, achieving high SOC is necessary. In this case, one could do a potential hold cycle after, say, every 30 cycles of galvanostatic cycling. And since the increase of AG for anthrone formation is more significant for pH tuning than control SOC and also the beneficial time per cycle for SOC management (only high SOC time e.g., 90%˜99% SOC) is far shorter than that of pH tuning (whole cycle time, e.g., 1%˜99% SOC), SOC management is less effective than strong pH method to improve anthraquinone cycling stability. Similarly, we could conclude that the capacity fade rate will decrease as time passed if anthrone oxidation is avoided because AG for anthrone formation increase as the accumulation of anthrone. Since the entropy change is positive for the disproportionation reaction, it is suggested to avoid both the full SOC state and high temperature operation.

### CONCLUSION

In summary, we synthesized three anthraquinone derivatives, carbon-linked, nitrogen-linked and oxygen-linked anthraquinones. The nitrogen-linked anthraquinone showed the lowest redox potential due to its strongest electron donating effect. It is synthesized from inexpensive precursors with a one-step N-alkylation method; therefore, the mass production cost might be very low. Although with four negative charges and high coulombic repulsion, its cycling performance is bad at neutral condition with a capacity fade rate of 2.6%/day. The capacity fade rate decreased by two orders of magnitude at pH 14, making it to be one of the most stable redox organics ever reported. The great difference in anthraquinone cycling stability at different pH values is explained by considering the thermodynamics of the numerous chemical and electrochemical reactions available to the system. This work shows the significant improvements that can be made with a better understanding of the capacity fade mechanism and its thermodynamics, and it shows the great potential of organics synthesis towards low-cost and stable electrolytes for AORFBs.

Other embodiments are in the claims.

What is claimed is:

1. A compound of formula (I): 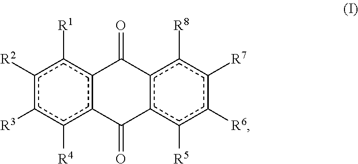
or an ion, salt, or hydroquinone thereof, where at least one of R¹, R², R³, R⁴, R⁵, R⁶, R⁷ and R⁸ is —N(R_{b})₂ or —N(R_{c})₂ wherein each R_{b} is independently an optionally substituted C₁₋₆ alkyl and each R_{c} is independently H or an optionally substituted C₁₋₆ alkyl, provided that when one R_{c} is H, the other R_{c} bound to the same N is an optionally substituted C₁₋₆ alkyl that is not CH₂CH₂C(═O)OH; wherein at least one R_{b} or R_{c} is an optionally substituted C₁₋₆ alkyl comprising a —S(═O)₂Rₐ; —S(═O)₂ORₐ; —OS(═O)₂ORₐ; —P(═O)Rₐ₂; or P(═O)(ORₐ)₂ substituent or is an optionally substituted C₂₋₆ alkyl comprising a —C(═O)ORₐ substituent; and each remaining of R¹, R², R³, R⁴, R⁵, R⁶, R⁷ and R⁸ is independently selected from H; halo; optionally substituted C₁₋₆ alkyl; oxo; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —SRₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; and —P(═O)(ORₐ)₂; or any two adjacent groups selected from R⁸, R⁷, R⁶, and R⁵ are joined to form an optionally substituted 3-6 membered ring, wherein each Rₐ is independently H; optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group.

2. The compound of claim 1, wherein any two or three of R¹, R², R³, R⁴, R⁵, R⁶, R⁷ and R⁸ are —N(R_{b})₂.

3. The compound of claim 1 or 2, wherein each R_{b} is independently selected from a C₁₋₆ alkyl group substituted with S(═O)₂OH, OS(═O)₂OH, or C(═O)OH, such as —CH₂S(═O)₂OH, —CH₂CH₂S(═O)₂OH, —CH₂CH₂CH₂CH₂S(═O)₂OH, —CH₂CH₂CH₂CH₂CH₂S(═O)₂OH, —CH₂CH₂CH₂CH₂CH₂CH₂S(═O)₂OH, —CH(CH₃)OS(═O)₂OH, —CH₂CH(CH₃)OS(═O)₂OH, —CH₂CH₂CH(CH₃)OS(═O)₂OH, —CH₂CH₂CH₂CH(CH₃)OS(═O)₂OH, —CH₂CH₂CH₂CH₂CH(CH₃)OS(═O)₂OH, —CH₂CH₂CH₂CH₂CH₂CH(CH₃)OS(═O)₂OH, —CH₂OS(═O)₂OH, —CH₂CH₂OS(═O)₂OH, —CH₂CH₂CH₂OS(═O)₂OH, —CH₂CH₂CH₂CH₂OS(═O)₂OH, —CH₂CH₂CH₂CH₂CH₂OS(═O)₂OH, —CH₂CH₂CH₂CH₂CH₂CH₂OS(═O)₂OH, —C(═O)OH, —CH₂C(═O)OH, —CH₂CH₂C(═O)OH, —CH₂CH₂CH₂C(═O)OH, —CH₂CH₂CH₂CH₂C(═O)OH, or —CH₂CH₂CH₂CH₂CH₂C(═O)OH, or a salt or ion thereof.

4. The compound of any one of claims 1-3, wherein the C₁₋₆ alkyl group is a C₃₋₆ alkyl group.

5. The compound of claim 1 or 2, wherein the compound of formula (I) is: 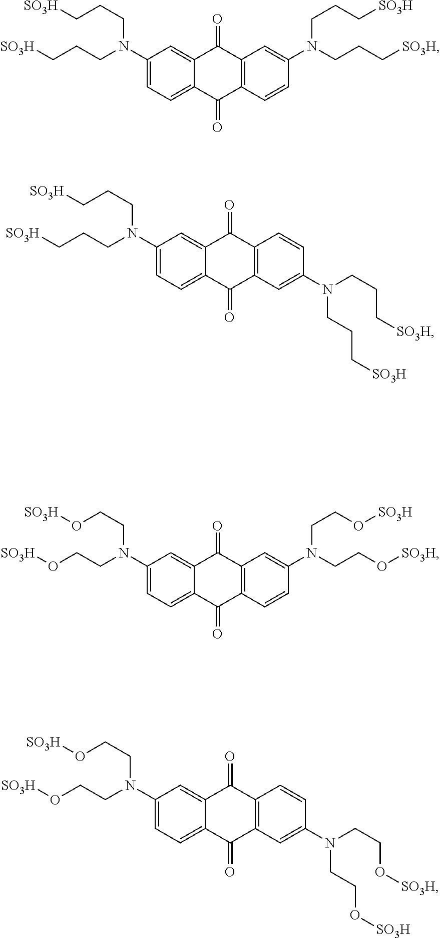 [SMILES: O=C1c2ccc(N(CCCS(=O)(=O)O)CCCS(=O)(=O)O)cc2C(=O)c2cc(N(CCCS(=O)(=O)O)CCCS(=O)(=O)O)ccc21 ; O=C1c2ccc(N(CCCS(=O)(=O)O)CCCS(=O)(=O)O)cc2C(=O)c2ccc(N(CCCS(=O)(=O)O)CCCS(=O)(=O)O)cc21 ; O=C1c2ccc(N(CCOS(=O)(=O)O)CCOS(=O)(=O)O)cc2C(=O)c2cc(N(CCOS(=O)(=O)O)CCOS(=O)(=O)O)ccc21 ; O=C1c2ccc(N(CCOS(=O)(=O)O)CCOS(=O)(=O)O)cc2C(=O)c2ccc(N(CCOS(=O)(=O)O)CCOS(=O)(=O)O)cc21] 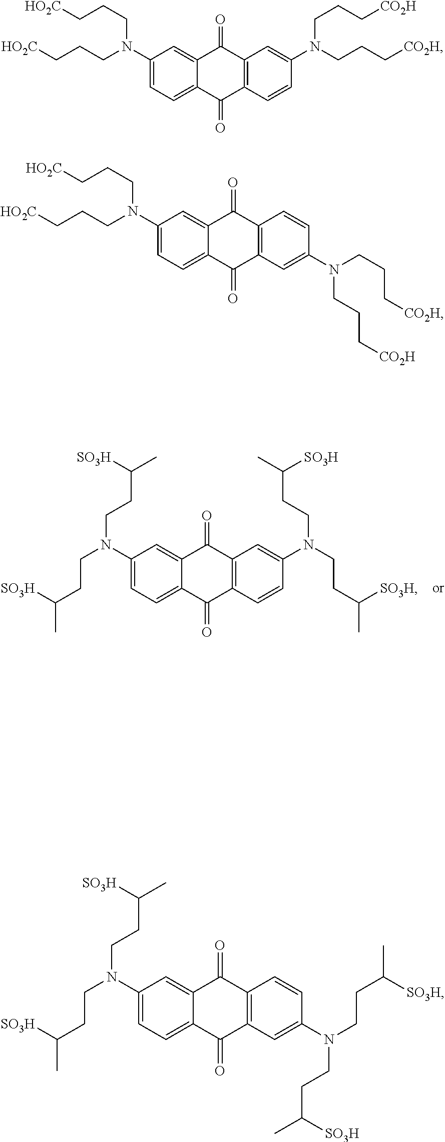 [SMILES: O=C(O)CCCN(CCCC(=O)O)c1ccc2c(c1)C(=O)c1cc(N(CCCC(=O)O)CCCC(=O)O)ccc1C2=O ; O=C(O)CCCN(CCCC(=O)O)c1ccc2c(c1)C(=O)c1ccc(N(CCCC(=O)O)CCCC(=O)O)cc1C2=O ; CC(CCN(CCC(C)S(=O)(=O)O)c1ccc2c(c1)C(=O)c1cc(N(CCC(C)S(=O)(=O)O)CCC(C)S(=O)(=O)O)ccc1C2=O)S(=O)(=O)O ; "or" ; CC(CCN(CCC(C)S(=O)(=O)O)c1ccc2c(c1)C(=O)c1ccc(N(CCC(C)S(=O)(=O)O)CCC(C)S(=O)(=O)O)cc1C2=O)S(=O)(=O)O]
or an ion, salt, or hydroquinone thereof.

6. The compound of claim 1, wherein the compound of formula (I) is produced by reacting 2,6-diaminoanthraquinone or 2,7-diaminoanthraquinone with 1,3-propanesultone, 1,3,2-dioxathiolane 2,2-dioxide, 3-methyl-1,2-oxathiolane 2,2-dioxide, or γ-butyrolactone.

7. The compound of claim 1, wherein the compound of formula (I) is produced by reacting 2,6-diaminoanthraquinone or 2,7-diaminoanthraquinone with a mixture of at least two of 1,3-propanesultone, 1,3,2-dioxathiolane 2,2-dioxide, or 3-methyl-1,2-oxathiolane 2,2-dioxide.

8. The compound of any one of claims 1-7, wherein the compound of formula (I) is a salt comprising a lithium cation, a potassium cation, a cesium cation, or an organic cation.

9. A method of producing a compound of formula (I), comprising reacting an aminoanthraquinone with 1,3-propanesultone, 1,3,2-dioxathiolane 2,2-dioxide, 3-methyl-1,2-oxathiolane 2,2-dioxide, γ-butyrolactone, or a combination thereof.

10. The method of claim 9, wherein the aminoanthraquinone is 2,6-diaminoanthraquinone or 2,7-diaminoanthraquinone.

11. A battery comprising first and second electrodes, wherein the first electrode is in contact with a posolyte and/or the second electrode is in contact with a negolyte, wherein the posolyte and/or negolyte comprises a compound of formula (I).

12. The battery of claim 11, further comprising a positive and a negative redox active species, and the compound of formula (I) is a redox mediator.

13. The battery of claim 11, wherein the battery is a redox flow battery or a redox targeting battery.

14. The battery of claim 13, wherein the redox flow battery comprises a redox active species which is or comprises bromine, chlorine, iodine, molecular oxygen, vanadium, chromium, cobalt, iron, aluminum, manganese, cobalt, nickel, copper, or lead.

15. A method of storing energy by oxidizing and/or reducing a compound of formula (I): 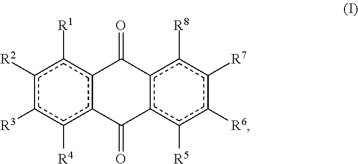
or an ion, salt, or hydroquinone thereof, where at least one of R¹, R², R³, R⁴, R⁵, R⁶, R⁷ and R⁸ is —N(R_{b})₂ or —N(R_{c})₂ wherein each R_{b} is independently an optionally substituted C₁₋₆ alkyl and each R_{c} is independently H or an optionally substituted C₁₋₆ alkyl, provided that when one R_{c} is H, the other R_{c} bound to the same N is an optionally substituted C₁₋₆ alkyl that is not CH₂CH₂C(═O)OH; and each remaining of R¹, R², R³, R⁴, R⁵, R⁶, R⁷ and R⁸ is independently selected from H; halo; optionally substituted C₁₋₆ alkyl; oxo; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN; —NO₂; —ORₐ; —SRₐ; —N(Rₐ)₂; —C(═O)Rₐ; —C(═O)ORₐ; —S(═O)₂Rₐ; —S(═O)₂ORₐ; —OS(═O)₂ORₐ; —P(═O)Rₐ₂; and —P(═O)(ORₐ)₂; or any two adjacent groups selected from R⁸, R⁷, R⁶, and R⁵ are joined to form an optionally substituted 3-6 membered ring, wherein each Rₐ is independently H; optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; an oxygen protecting group; or a nitrogen protecting group.

16. The method of claim 15, wherein the compound of formula (I) is a negolyte of a redox flow battery.

17. The method of claim 16, wherein the compound of formula (I) is a redox mediator.
